(19) United States
(12) Patent Application Publication
(10) Pub. No.: US 2025/0262449 A1
(43) Pub. Date: Aug. 21, 2025

(54) PHOTOBIOMODULATION THERAPY DEVICES AND METHODS OF USE

(71) Applicant: Joovv, Inc., Carson City, NV (US)

(72) Inventors: Greg Pfiffner, Dayton, NV (US); Bryan Chang, New Taipei City (TW)

(21) Appl. No.: 19/055,903

(22) Filed: Feb. 18, 2025

Related U.S. Application Data
(60) Provisional application No. 63/554,498, filed on Feb. 16, 2024.

(51) Int. Cl. A61N 5/06; A61B 17/00

(52) U.S. Cl. CPC A61N 5/0616; A61B 2017/00154; A61B 2017/00221; A61N 2005/0626; A61N 2005/0632; A61N 2005/0643; A61N 2005/0652; A61N 2005/0659; A61N 2005/0663; A61N 2005/0664

(57) ABSTRACT

The disclosure includes a light therapy device comprising a flexible textile material, a flexible printed circuit board (PCB) assembly coupled to the flexible textile material, a plurality of light emitting diodes (LEDs) electrically coupled to the flexible PCB assembly, wherein the plurality of LEDs is arranged and configured to emit at least one of red light and near-infrared light, and a controller electrically coupled to the flexible PCB assembly, configured to control emission of the at least one of red light and near-infrared light from the plurality of LEDs.

### CROSS REFERENCE TO RELATED APPLICATION

This application claims priority to U.S. Provisional Patent Application No. 63/554,498 filed Feb. 16, 2024, the entirety of which is incorporated by reference.

### FIELD OF THE INVENTION

The present invention generally relates to red light therapy and, more particularly, relates to flexible textile material that emits therapeutic red light.

### BACKGROUND OF THE INVENTION

Red light therapy (RLT) involves having red or near-infrared (NIR) light wavelengths emitted directly through the skin. Red or NIR light can be absorbed into the skin to a depth of about eight to 10 millimeters, at which point it has positive effects on cellular energy and multiple nervous system and metabolic processes.

Red light therapy has shown promise for treating symptoms of joint pain or osteoarthritis due to aging, those caused by cancer treatments like chemotherapy or radiation, hair loss, wounds or incisions, acne, wrinkles and skin discoloration, chronic muscular pain, neurological damage, and tissue damage (often at the root of tears, sprains or pulls).

Many of the current light therapy devices and lighting arrays are designed for desktop use or are complete chambers that the user enters. The vast majority of RLT devices on the market are either hand-held (very small treatment area) or a countertop unit that treats the face. Tanning salons offer red light treatment sessions in converted tanning beds using fluorescent circuits but the cost and size make these devices impractical for home use. In addition, the fluorescent circuits are very inefficient so the intensity of the light compromises the effectiveness of these devices.

Each of these prior-art devices rely on built-in structural elements that inherently restrict flexibility. The incorporation of rigid internal frameworks has been a standard feature, a design choice that compromises the device's ability to mold to the body's natural form. These internal supports, while sometimes necessary for other functional reasons, create a barrier to achieving the full range of motion and comfort required for intimate, ergonomic applications.

Therefore, a lighting apparatus/system is needed that can be better adapted for use with various parts of the body of a user than that which, up to now, has not been available. What is needed is a novel design that is entirely devoid of internal structural impediments and that allows the light-emitting device to adapt seamlessly to various body shapes.

### SUMMARY OF THE INVENTION

The disclosure includes a light therapy device comprising a flexible textile material, a flexible printed circuit board (PCB) assembly coupled to the flexible textile material, a plurality of light emitting diodes (LEDs) electrically coupled to the flexible PCB assembly, wherein the plurality of LEDs is arranged and configured to emit at least one of red light and near-infrared light, and a controller electrically coupled to the flexible PCB assembly, configured to control emission of the at least one of red light and near-infrared light from the plurality of LEDs.

In some embodiments, the flexible textile material comprises a front cover and a back cover removably coupled to the front cover via at least one attachment mechanism. The front cover may be configured to receive the flexible PCB assembly and may include a semi-transparent material configured to diffuse at least a portion of the at least one of red light and near-infrared light emitted by the plurality of LEDs. In some embodiments, the flexible PCB assembly and the plurality of LEDs are detachably coupled to the flexible textile material. The flexible textile material may be machine washable. In some embodiments, the flexible textile material is capable of diffusing heat generated by the plurality of LEDs.

The controller may include a plurality of input buttons. In some embodiments, the plurality of input buttons includes a play/pause button configured to start and stop emission of the at least one of red light and near-infrared light from the plurality of LEDs, a mode button configured to select a light emission mode of the plurality of LEDs, and a time button configured to select a treatment time. The light emission mode is selected from the group consisting of red-light emission only, near-infrared light emission only, red and near-infrared light emission, and a recovery mode. In some embodiments, the recovery mode comprises substantially continuous red-light emission with pulsed near-infrared light emission. The controller may comprise a power button configured to at least one of enter and exit a standby state and a pairing button configured to at least one of turn on and off local area network pairing with a third-party device.

In some embodiments, the light therapy device includes a connector coupled to the flexible textile material and electrically coupled to the flexible PCB assembly, wherein the connector is configured to receive a cable coupled to the controller, thereby electrically coupling the controller to the flexible PCB assembly. The controller may be electrically coupled to an external power supply unit configured to provide power to the light therapy device.

In some embodiments, the controller comprises a display screen. The display screen may be configured to display information selected from the group consisting of treatment time, light emission mode, local area network pairing status, and treatment start/stop status. In some embodiments, the controller comprises a touch screen configured to receive a user input.

The flexible PCB assembly may comprise a plurality of flexible printed circuit boards (PCBs). In some embodiments, the light therapy device further comprises a connector coupled to each flexible PCB of the plurality of flexible PCBs.

The flexible textile material may define a length of about 1840 millimeters and a width of about 915 millimeters.

In some embodiments, the flexible textile material comprises a first side coupled to the flexible PCB assembly and a second side located opposite the first side. The controller may be detachably coupled to the second side of the flexible textile material. In some embodiments, the controller comprises a built-in rechargeable battery configured to provide power to the light therapy device. The controller may comprise a light emitting diode (LED) configured to indicate a status of a charge level of the built-in rechargeable battery.

In some embodiments, the controller comprises a plurality of input buttons. The plurality of input buttons may comprise a time button configured to select a predetermined treatment time defining 10, 20, or 30 minutes.

In some embodiments, the flexible textile material defines a length of about 620 millimeters and a width of about 220 millimeters.

The disclosure includes a wearable system comprising a plurality of light-emitting diode (LED) devices coupled to a wearable device, one or more sensors coupled to the wearable device, and a controller coupled to the plurality of LED devices. In some embodiments, the system includes a transceiver being configured to couple the controller to a wireless network. The plurality of LED devices may comprise one or more arrays of LED devices wherein LED devices with an associated array may be configured to emit light within a wavelength of between 660 nanometers and 850 nanometers. In some embodiments, the transceiver is included in a smartphone or a tablet. Ones of the plurality of LED devices may be configured to emit light in a wavelength range of between 600 nanometers and 630 nanometers.

The disclosure includes a method for providing light therapy, comprising receiving one or more instructions from a controller, operating a plurality of light-emitting diodes (LEDs) coupled to a wearable device, in accordance with the one or more instructions received from the controller, the plurality of LEDs providing light therapy at light wavelengths between about 630 nanometers and 660 nanometers, measuring one or more parameters associated with the wearable device or a user of the wearable device, and transmitting data, including one or more of the one or more parameters measured, to a remote location. In some embodiments, the one or more parameters are selected from the group consisting of temperature, power output, user temperature, user heart rate and a combination thereof.

Ones of the plurality of LEDs may operate to provide light at a wavelength of between about 630 nanometers and 660 nanometers. In some embodiments, ones of the plurality of LEDs operate to provide light at a wavelength of between about 800 nanometers and 900 nanometers. Ones of the plurality of LEDs may operate to provide light at a wavelength of between about 600 nanometers and 750 nanometers. In some embodiments, ones of the plurality of LEDs operate to provide light at a wavelength of between about 900 nanometers and 1 millimeter.

Ones of the plurality of LEDs may be operable to produce light at wavelengths selected from the group consisting of light at a wavelength of between about 630 nanometers and 660 nanometers, light at a wavelength of between about 800 nanometers and 900 nanometers, light at a wavelength of between about 600 nanometers and 750 nanometers, light at a wavelength of between about 900 nanometers and 1 millimeter and a combination thereof.

In some embodiments, the one or more instructions from the controller include instructions to selectively turn on or turn off selected LEDs from the plurality of LEDs producing light at selected wavelengths. The method may include storing the data at the remote location.

The disclosure includes a computer-readable, non-transitory, programmable product, comprising code, executable by a processor, for causing the processor to do the following: cause reception of instructions from a remote location, operate a plurality of arrays of light-emitting diodes (LEDs) coupled to a wearable device, according to the instructions, receive sensor data associated with

the wearable device, and cause transmission of the sensor data to a remote location. In some embodiments, the instructions include operating a plurality of LEDs in the plurality of arrays in a manner that delivers light over a range of wavelengths. The range of wavelengths may be between about 600 nanometers and 750 nanometers. In some embodiments, the range of wavelengths is between about 800 nanometers and 900 nanometers. The range of wavelengths may be between about 900 nanometers and 1 millimeter.

The light therapy device also includes a flexible textile material. The device also includes a flexible substrate assembly coupled to and interior of the flexible textile material; a flexible textile cable assembly coupled to an interior of the flexible textile material; a plurality of light emitting diodes (LEDs) electrically coupled to the flexible substrate assembly, where the plurality of LEDs is arranged and configured to emit at least one of red light and near-infrared light; and a controller electrically coupled to the flexible textile cable assembly and the flexible substrate assembly, configured to control emission of the at least one of red light and near-infrared light from the plurality of LEDs.

Implementations may include one or more of the following features. The light therapy device where the flexible textile material may include: at least one attachment mechanism removably coupling the flexible substrate assembly and the flexible textile cable assembly to the interior of the flexible textile material. The at least one attachment mechanism is a non-conductive snap structure. The flexible textile material may include: a front cover of a semi-transparent material configured to diffuse at least a portion of the at least one of red light and near-infrared light emitted by the plurality of LEDs. The flexible textile material is at least partially metallic and is capable of diffusing heat generated by the plurality of LEDs. The controller may include: a select mode button configured to select a light emission mode of the plurality of LEDs; a help button configured to open up help menus; a settings button configured to open up a settings menu; a play button configured to start/stop/pause emission mode of the plurality of LEDs; and a time button configured to select a treatment time. The light emission mode may include preset modes and a custom mode selection, where the preset modes include red and near-infrared light in continuous and pulsed waveform at preset time durations and where the custom mode selection includes red and near-infrared light in continuous and pulsed waveform at customizable time durations. The recovery mode may include: substantially continuous red-light emission with pulsed near-infrared light emission. The controller may include: a power button configured to at least one of enter and exit a standby state; and a pairing button configured to at least one of turn on and off local area network pairing with a third-party device. The textile cable assembly is electrically coupled to the substrate assembly and the connector is configured to receive a cable coupled to a controller docking station, thereby electrically coupling the controller docking station to the nonwoven coated substrate assembly. The controller may include: a display screen configured to display information indicating at least one of treatment time, light emission mode, local area network pairing status, and treatment start/stop status. The flexible PCB assembly may include: a nonwoven coated substrate; a plurality of flexible printed circuit boards (PCBs) on the nonwoven coated substrate; and a plurality of electrically conductive connectors each attached to one of the plurality of flexible PCBs. The controller may include a built-in rechargeable battery configured to provide power to the light therapy device.

The wearable led treatment system also includes a flexible substrate assembly; a flexible textile cable assembly physically and electrically coupled to the flexible substrate assembly; a plurality of light emitting diodes (LEDs) physically and electrically coupled to the flexible substrate assembly, where the plurality of LEDs is arranged and configured to emit at least one of red light and near-infrared light; a power source electrically coupled to the flexible textile cable assembly; and a flexible textile cover including: a front panel having a semi-transparent portion configured to diffuse at least a portion of the at least one of red light and near-infrared light emitted by the plurality of LEDs; and a back panel with fasteners configured to removably couple and decouple the flexible textile cable assembly to the flexible textile cover.

The wearable led treatment system where the back panel is made of a metallic material and is capable of diffusing heat generated by the plurality of LEDs The fasteners are non-conductive snap structures. The wearable led treatment system may include: a controller electrically coupled to the flexible textile cable assembly and the flexible substrate assembly, configured to control emission of the at least one of red light and near-infrared light from the plurality of LEDs The wearable led treatment system may include: a transceiver configured to couple the controller to a wireless network. The transceiver is included in a smartphone or a tablet. Ones of the plurality of LEDs are operable to produce light at wavelengths selected from the group may include of light at a wavelength of between about 630 nanometers and 660 nanometers, light at a wavelength of between about 800 nanometers and 900 nanometers, light at a wavelength of between about 600 nanometers and 750 nanometers, light at a wavelength of between about 900 nanometers and 1 millimeter and a combination thereof.

The disclosure includes a computer-readable, non-transitory, programmable product wherein the code further causes the processor to produce training inputs for a training model used to produce artificial intelligence-generated instructions for control of the wearable device and associated functionality.

The foregoing, and other features and advantages of the invention, will be apparent from the following, more particular description of the preferred embodiments of the invention, the accompanying drawings, and the claims.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 illustrates a light therapy device lying on a user, according to some embodiments.

FIG. 2A illustrates a font view of the light therapy device, according to some embodiments.

FIG. 2B illustrates a back view of the light therapy device, according to some embodiments.

FIG. 3 illustrates a front view of the light therapy device coupled to a controller, according to some embodiments.

FIG. 4A illustrates a front view of the controller, according to some embodiments.

FIG. 4B illustrates a back view of the controller, according to some embodiments.

FIG. 5A illustrates a top view of the controller, according to some embodiments.

FIG. 5B illustrates a bottom view of the controller, according to some embodiments.

FIG. 6 illustrates a light therapy device lying on a user, according to some embodiments.

FIG. 7A illustrates a front view of the light therapy device, according to some embodiments.

FIG. 7B illustrates a back view of the light therapy device, including a controller, according to some embodiments.

FIG. 8A illustrates a front view of the controller, according to some embodiments.

FIG. 8B illustrates a back view of the controller, according to some embodiments.

FIG. 9A illustrates a top view of the controller, according to some embodiments.

FIG. 9B illustrates a bottom view of the controller, according to some embodiments.

FIG. 10 illustrates a block diagram representing a wearable light therapy device, according to some embodiments.

FIG. 11 illustrates a flowchart of operating the wearable light therapy device, according to some embodiments.

FIG. 12 illustrates a plan view of a flexible substrate assembly, according to some embodiments.

FIG. 13 illustrates a plan view of a flexible textile cable assembly, according to some embodiments.

FIG. 14 illustrates a partial view of flexible substrate assembly being coupled to a flexible textile cable assembly, according to some embodiments.

FIG. 15 illustrates a fully assembled flexible light-emitting blanket assembly, according to some embodiments.

FIG. 16 illustrates the internal wiring of an exemplary one of the anchor strips of the flexible textile cable assembly, according to some embodiments.

FIG. 17 illustrates a docking station for a controller, according to some embodiments.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

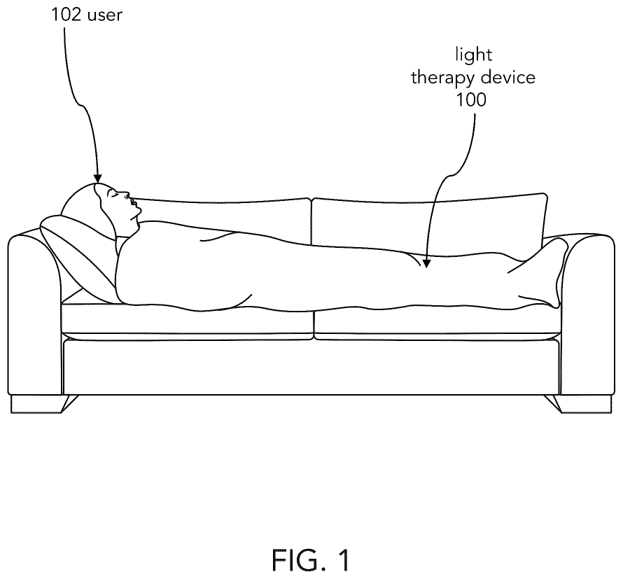

FIG. 1 illustrates a light therapy device 100 lying on a user 102. In some embodiments, the light therapy device 100 comprises a blanket-type article configured to lie directly on the user 102 while providing light therapy. The light therapy device 100 may also include a wearable garment, such as, but not limited to, a robe, jacket, sweatshirt, pants, shorts, or dress. As shown in FIG. 1, the light therapy device 100 may define a length sufficient to cover a large portion of the body of the user 102.

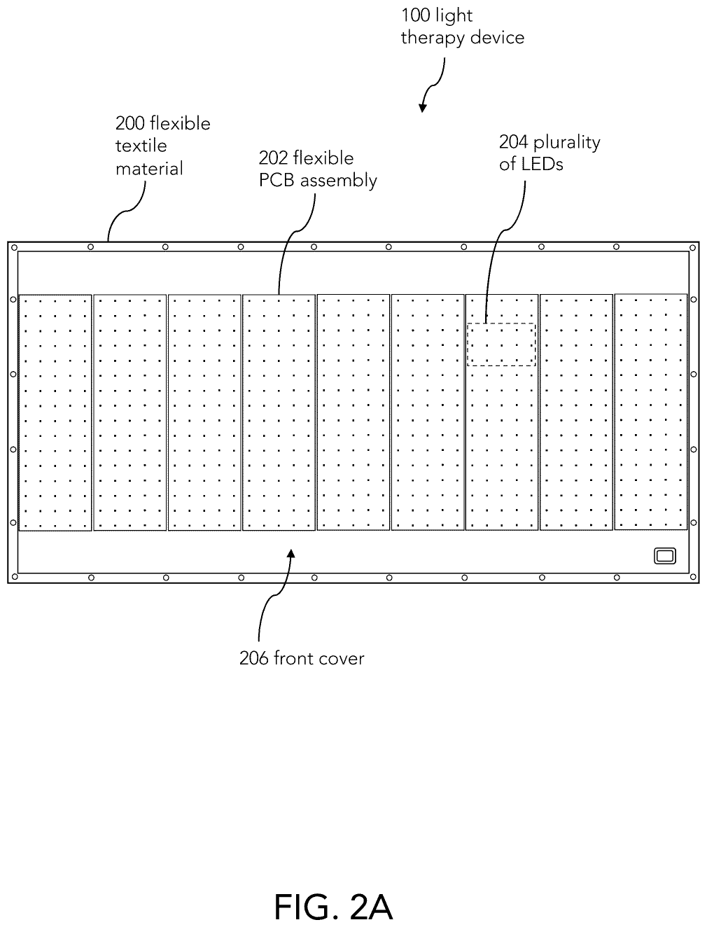

FIG. 2A illustrates a front view of the light therapy device 100, including a flexible textile material 200 and a flexible PCB assembly 202. In some embodiments, the flexible PCB assembly 202 is electrically coupled to a plurality of LEDs 204, and the plurality of LEDs 204 are arranged and configured to emit at least one of red light and near-infrared (NIR) light, i.e., red light and/or NIR. It should be noted that FIG. 2A includes a dashed box around several LEDs, labeled as the plurality of LEDs 204. The plurality of LEDs 204 can vary in number and may include every small square shown on the flexible PCB assembly 202.

In some embodiments, the flexible textile material 200 includes a front cover 206, as shown in FIG. 2A. The front cover 206 may be detachably coupled to the flexible PCB assembly 202. For example, in some embodiments, the front cover 206 includes one or multiple pockets configured to receive the flexible PCB assembly 202. The flexible PCB assembly 202 may be coupled to the front cover 206 via other methods, such as hook and loop fastener, a plurality of snaps, a plurality of zippers, or any other similar method. In some embodiments, the front cover 206 comprises a semi-transparent material configured to diffuse at least a portion of the light emitted by the plurality of LEDs 204. The front cover 206 may be comprised of mesh or a similar material. The light therapy device 100 may include an additional layer of material between the front cover 206 and the flexible PCB assembly 202 to provide an air gap and additional diffusing of the light emitted by the plurality of LEDs 204.

FIG. 2B illustrates a back view of the light therapy device 100. As shown in FIG. 2B, in some embodiments, the flexible textile material 200 includes a back cover 208 in addition to the front cover 206 shown in FIG. 1. The back cover 208 may be detachably coupled to the front cover 206 via at least one attachment mechanism 210. In some embodiments, the at least one attachment mechanism 210 is a plurality of snaps, buttons, or the like. The at least one attachment mechanism 210 may be one or multiple zippers, hook and loop fastener, or any number of similar mechanisms configured for detachable coupling.

In some embodiments, the back cover 208 is a non-translucent fabric. The back cover 208 may include at least one pocket configured to receive the flexible PCB assembly 202, such that the flexible PCB assembly 202 may be coupled to the back cover 208 and surrounded by the front cover 206. In some embodiments, the flexible textile material 200, including the front cover 206 and the back cover 208, is made of machine-washable material. The front cover 206 and the back cover 208 may be capable of diffusing heat generated by the plurality of LEDs 204. The light therapy device 100 may include at least one designated heatsink, in addition to or instead of using a heat-dissipating flexible textile material 200. In some embodiments, the flexible textile material 200, combined with the flexible PCB assembly 202, creates a non-shape-retaining blanket or similar article configured to be placed on a user to provide light therapy. FIG. 2B also includes a product label 212 in the lower right corner of the back cover 208. In some embodiments, the product label 212 is configured to include information such as brand name, care instructions for the flexible textile material 200, product warnings, and the like.

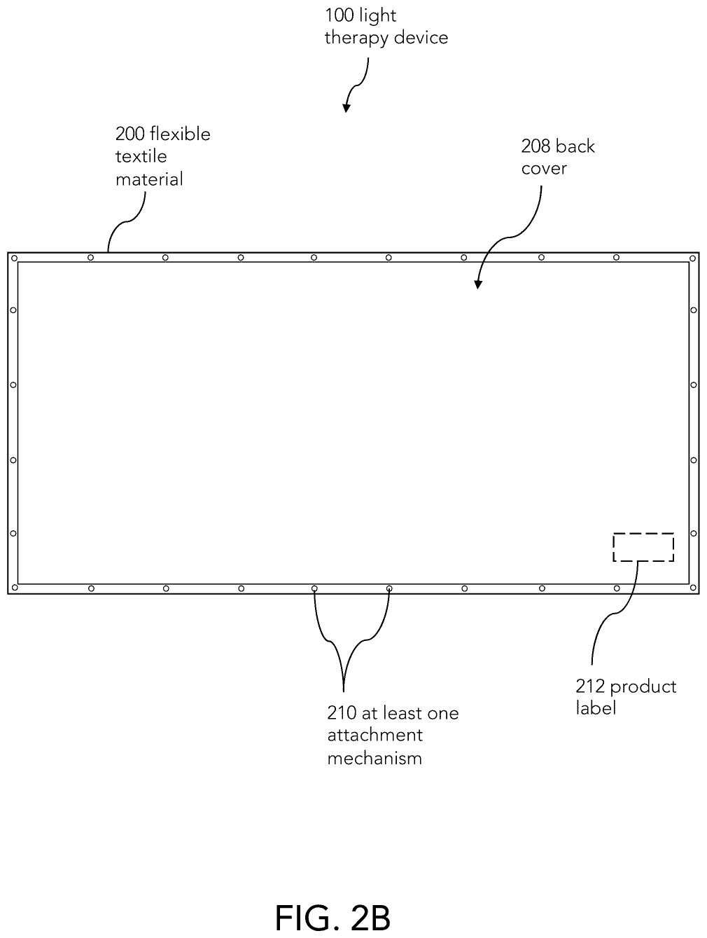

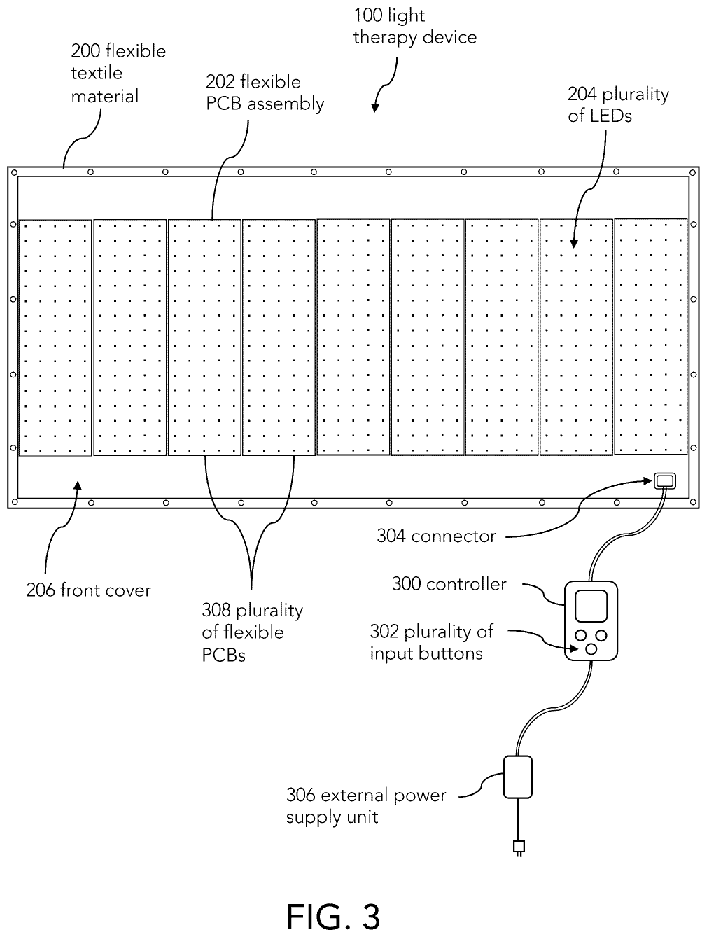

FIG. 3 again illustrates a front view of the light therapy device 100 and includes a controller 300 coupled to the light therapy device 100. In some embodiments, the controller 300 is electrically coupled to the flexible PCB assembly 202 and is configured to control emission of red light and/or NIR light emitted from the plurality of LEDs 204. The controller 300 may include a plurality of input buttons 302, which will be discussed further with reference to FIG. 4A.

In some embodiments, the light therapy device 100 includes a connector 304 located on the flexible textile material 200. The connector 304 may be electrically coupled to the flexible PCB assembly 202 and configured to receive a cable coupled to the controller 300, in order to thereby electrically and communicatively couple the flexible PCB assembly 202 to the controller 300. In some embodiments, the controller 300 is also coupled to an external power supply unit 306. The external power supply unit 306 may be configured to provide power to the controller 300 and the flexible PCB assembly 202, including the plurality of LEDs 204. In some embodiments, the external power supply unit 306 comprises a 300 W power supply unit.

FIG. 3 also includes a plurality of flexible PCBs 308. In some embodiments, the flexible PCB assembly 202 comprises a plurality of flexible PCBs 308. As illustrated in FIG. 3, the light therapy device 100 may include a total of nine flexible PCBs in the plurality of flexible PCBs 308. Each flexible PCB of the plurality of flexible PCBs 308 may define a length of about 610 millimeters and a width of about 200 millimeters. In some embodiments, the light therapy device 100 includes a connector coupling each flexible PCB of the plurality of flexible PCBs 308 to one another. Once connected, the plurality of flexible PCBs 308 may be configured to operate as a unit-the flexible PCB assembly 202. The invention is in no way limited to nine flexible PCBs and can include more or less than nine. Similarly, the invention is in no way limited to dimensions of 610 millimeters for the length and/or 200 millimeters for the width and can be of dimensions that are larger or smaller than these exemplary numbers.

FIGS. 4A and 4B show front and back views, respectively, of the controller 300. In some embodiments, the controller 300 includes a screen 400, as illustrated in FIG. 4A. The screen 400 may be a display screen configured to show the treatment start/stop status 404, the countdown time 406, the local area network pairing status 408, among other possible information, such as the selected light mode 410 and treatment time 412, as shown in FIG. 4A. In some embodiments, the screen 400 comprises a touch screen configured to receive input from a user. The screen 400 may be configured to receive input and display information. In some embodiments, the screen 400 comprises a color LCD display. The screen 400 may be an OLED display.

As shown in FIG. 3, the controller 300 may include a plurality of input buttons 302. In some embodiments, as shown in FIG. 4A, the plurality of input buttons 302 includes a mode button 402a, a play/pause button 402b, and a time button 402c. The mode button 402a may be configured to select a light emission mode of the plurality of LEDs 204, the play/pause button 402b may be configured to start and stop emission of red light and/or NIR light from the plurality of LEDs 204, and the time button 402c may be configured to select a treatment time. In some embodiments, the plurality of input buttons 302 correspond with the information displayed on the screen 400. For example, pressing the mode button 402a changes the selected light mode 410 shown on the screen 400. Similarly, whether a treatment session is running or paused, as selected by the play/pause button 402b, may be indicated by the treatment start/stop status 404 on the screen 400.

In some embodiments, pressing the time button 402c increases the treatment time 412, as displayed on the screen 400, by 1 minute increments, beginning at a 1 minute treatment session and ranging up to a 30 minute treatment session. If the time button 402c is pressed again after reaching a maximum 30 minute treatment session, the treatment time will go back to 1 minute. A default treatment time of 10 minutes may be selected upon turning on the light therapy device 100. The countdown time 406 on the screen 400 may be configured to show the time remaining in a treatment session. For example, the treatment session demonstrated in FIG. 4A comprises a 10 minute session with 8 minutes and 43 seconds remaining.

In some embodiments, the light emission mode selected by the mode button 402 comprises one of red light emission only (as indicated by the selected light mode 410 in FIG. 4A), NIR light emission only, red and NIR light emission, and a recovery mode, which may also be called a “Recovery Plus Mode.” Pressing the mode button 402 may cycle through these four light emission mode options, as reflected by the selected light mode 410 on the screen 400. When the light therapy device 100 is arranged and configured to operate in the Recovery Plus Mode, the plurality of LEDs 204 may be configured to emit pulsed infrared light. In some embodiments, the plurality of LEDs 204 is configured to emit substantially continuous red light in the Recovery Plus Mode. The pulsed infrared light may be emitted at a single frequency. In some embodiments, the infrared light is pulsed at varying frequencies throughout the duration of the Recovery Plus Mode. “Varying” frequencies may comprise frequencies between 0 and 100 Hz, emitted at different duty cycle percentages. For example, the Recovery Plus Mode may include pulsing infrared light at 25 Hz and at 50% brightness for two minutes, increasing the frequency to 50 Hz at 75% brightness for two minutes, then reducing the frequency to 35 Hz and 40% brightness for two minutes.

The Recovery Plus Mode may comprise any combination of frequency and brightness per duty cycle. The infrared light may be pulsed for a certain amount of time; for example, the infrared light may be emitted for two seconds every five seconds. The infrared light may be pulsed for a shorter or longer duration than two seconds. In some embodiments, the Recovery Plus Mode comprises pulsing red light and substantially continuous infrared light. The Recovery Plus Mode may include pulsed or continuous emission of infrared and/or near-infrared light. The duration of a treatment session using Recovery Plus Mode may be selected by a user of the light therapy device 100 via the controller 300. The Recovery Plus Mode may help facilitate cellular recovery by encouraging the removal of waste products from cells during a “quench period” in the cellular recovery process. The light therapy device 100 may be configured to automatically shut off at the end of a Recovery Plus Mode session. A user may manually shut off the light therapy device 100. FIG. 4B shows a back view of the controller 300. In some embodiments, the controller 300 includes a recessed area 414, indicated by the dashed box in FIG. 4B. The recessed area 414 may be configured to receive a label with product information such as branding, use instructions, product warnings, and the like. The controller 300 may also include a plurality of screws 416 to hold the housing of the controller 300 together. In some embodiments, the housing of the controller 300 is made of ABS plastic and has an ingress protection (IP) 22 rating.

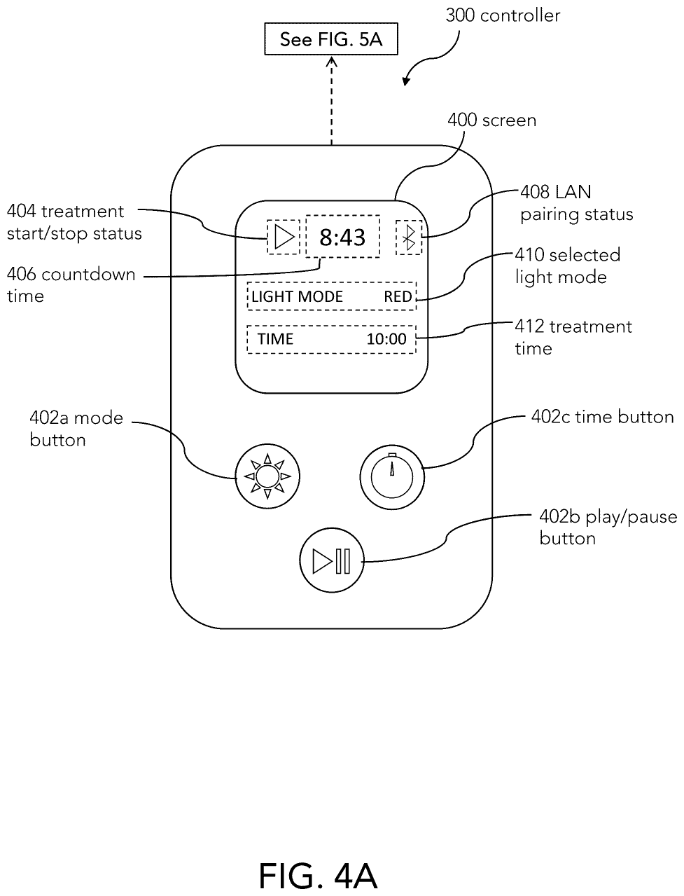

FIGS. 5A and 5B show top and bottom views, respectively, of the controller 300. In some embodiments, as shown in FIG. 5A, the controller 300 includes a power button 500 configured to turn the light therapy device 100 off or on, depending on the current state. The controller 300 may also include a pairing button 502 configured to enable a user to pair (or unpair) the light therapy device 100 with a third-party device via a local area network, such as Bluetooth®. In some embodiments, the light therapy device 100 is compatible with a mobile application configured to run on a third-party device such as a smartphone or tablet. The mobile application may operate similarly to the controller 300 in terms of starting or stopping a treatment session and/or selecting treatment time and/or light emission mode. The pairing button 502 may also turn on and/or off pairing for the light therapy device 100. For example, a single press of the pairing button 502 may turn on/off pairing by turning on or off access to the LAN. A press and hold of the pairing button 502 may initiate the pairing process, allowing the light therapy device 100 to “search” for a nearby third-party device for connection. In some embodiments, whether the light therapy device 100 is paired or not is reflected by the presence of an icon in the LAN pairing status 408 on the screen 400, as shown in FIG. 4A.

The controller 300 may also include a first input port 504, a second input port 506, and a third input port 508, as shown in FIGS. 5A and 5B. In some embodiments, the first input port 504 comprises a light device input port and is configured to receive the cable coupled to the connector 304 in the flexible textile material 200, as discussed with reference to FIG. 3. The second input port 506 may comprise a Mini-USB port for downloading and/or uploading firmware to the controller 300. In some embodiments, the third input port 508 comprises a power input, such as a 24V DC power input, to connect the controller 300 to the external power supply unit 306 shown in FIG. 3.

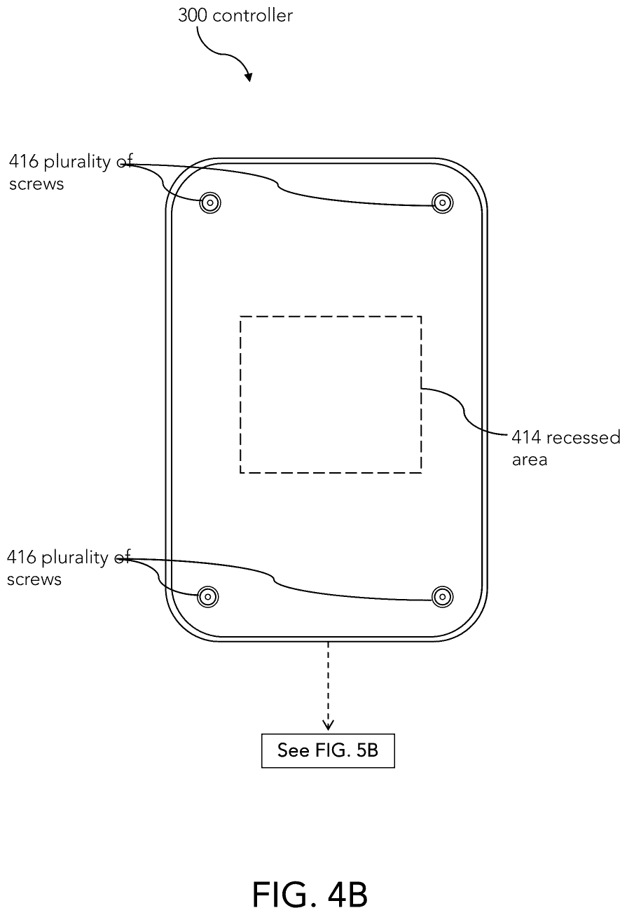

FIG. 6 illustrates a light therapy device 600 laying on a user 602. Similar to the light therapy device 100, in some embodiments, the light therapy device 600 comprises a blanket-type article configured to lay directly on the user 602 while providing light therapy. The light therapy device 600 may also comprise a wearable garment, such as, but not limited to, a robe, jacket, sweatshirt, pants, shorts, or dress. As compared to the light therapy device 100, the light therapy device 600 may define a length sufficient to cover a smaller portion of the body of the user 602.

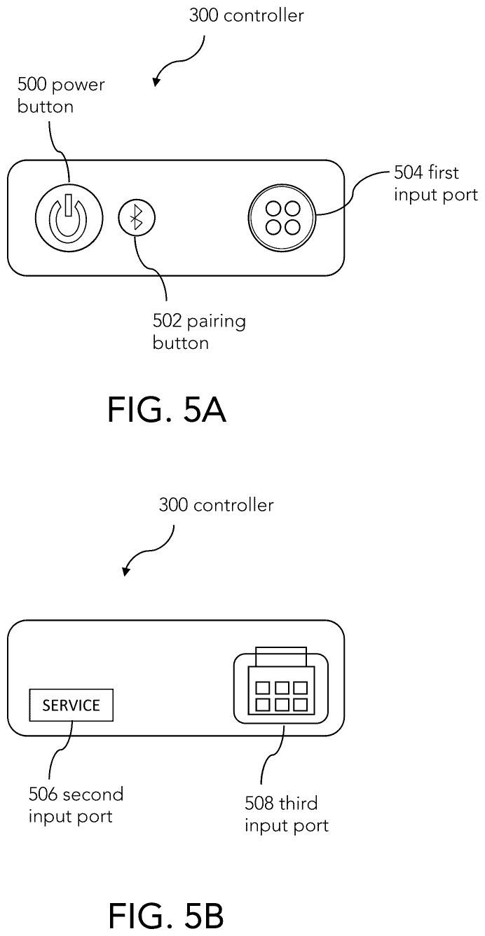

FIG. 7A shows a front view of the light therapy device 600, including a flexible textile material 700 and a flexible PCB assembly 702. In some embodiments, the light therapy device 600 is similar to the light therapy device 100, but with scaled-down dimensions. For example, the flexible textile material 200 of the light therapy device 100 may define a length of about 1840 millimeters and a width of about 915 millimeters, whereas the flexible textile material 700 of the light therapy device 600 may define a length of about 620 millimeters and a width of about 220 millimeters. In addition, where flexible PCB assembly 202 of the light therapy device 100 comprises a plurality of flexible PCBs 308, the flexible PCB assembly 702 of the light therapy device 600 may comprise a single flexible PCB, rather than multiple. The flexible PCB assembly 702 of the light therapy device 600 may be electrically coupled to a plurality of LEDs 704 configured to emit at least one of red light and NIR light, similar to the plurality of LEDs 204 of the light therapy device 100.

In some embodiments, the flexible textile material 700 includes a front cover 706, as shown in FIG. 7A. The front cover 706 may be detachably coupled to the flexible PCB assembly 702. For example, in some embodiments, the front cover 706 includes one or multiple pockets configured to receive the flexible PCB assembly 702. The flexible PCB assembly 702 may be coupled to the front cover 706 via other methods, such as hook and loop fastener, a plurality of snaps, a plurality of zippers, or any other similar method. In some embodiments, the front cover 706 comprises a semi-transparent material configured to diffuse at least a portion of the light emitted by the plurality of LEDs 704. The front cover 706 may be comprised of mesh or a similar material. The light therapy device 600 may include an additional layer of material between the front cover 706 and the flexible PCB assembly 702 to provide an air gap and additional diffusing of the light emitted by the plurality of LEDs 704.

FIG. 7B illustrates a back view of the light therapy device 600. As shown in FIG. 7B, in some embodiments, the flexible textile material 700 includes a back cover 710 in addition to the front cover 706 shown in FIG. 7A. The back cover 710 may be detachably coupled to the front cover 706 via at least one attachment mechanism 712. In some embodiments, the at least one attachment mechanism 712 comprises a plurality of snaps, buttons, or the like. The at least one attachment mechanism 712 may comprise one or multiple zippers, hook and loop fastener, or any number of similar mechanisms configured for detachable coupling.

In some embodiments, the back cover 710 comprises non-translucent fabric. The back cover 710 may include at least one pocket configured to receive the flexible PCB assembly 702, such that the flexible PCB assembly 702 may be coupled to the back cover 710 and surrounded by the front cover 706. In some embodiments, the flexible textile material 700, including the front cover 706 and the back cover 710, is comprised of machine-washable material. The front cover 706 and the back cover 710 may be capable of diffusing heat generated by the plurality of LEDs 704. The light therapy device 600 may include at least one designated heatsink, in addition to or instead of using a heat-dissipating flexible textile material 700. In some embodiments, the flexible textile material 700, combined with the flexible PCB assembly 702, creates a non-shape-retaining blanket or similar article configured to be placed on a user to provide light therapy. FIG. 7B also includes a product label 714 in the lower right corner of the back cover 710. In some embodiments, the product label 714 indicates a label configured to include information such as brand name, care instructions for the flexible textile material 700, product warnings, and the like.

FIG. 7B also includes a controller 708. In some embodiments, the controller 708 is detachably coupled to the back cover 710 of the light therapy device 600 and is electrically coupled to the flexible PCB assembly 702. The controller 708 may be configured to control emission of the at least one of red light and NIR light from the plurality of LEDs 704. Coupling of the controller 708 to the back cover 710 may allow for wireless use of the light therapy device 600.

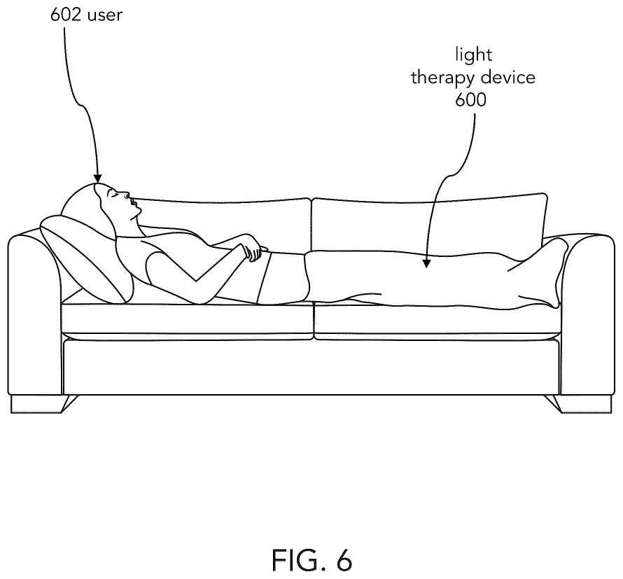

FIGS. 8A and 8B illustrate front and back views, respectively, of the controller 708. In some embodiments, the controller 708 comprises a built-in rechargeable battery 800 configured to provide power to the light therapy device 600. Though the battery 800 is indicated in FIG. 8A as a dashed box, it should be noted that the battery 800 may define a different shape or be larger or smaller than represented by the box. In some embodiments, the battery 800 comprises a lithium-ion battery. The battery 800 may comprise any suitable type of rechargeable battery. The controller 708 may also include an indicator LED 802 configured to indicate the status of the charge level of the battery 800. For example, the indicator LED 802 may be configured to emit different light colors based on the charge level of the battery 800 (e.g., green for a full charge, yellow for a medium charge, and red or orange for no or low charge).

The front of the controller 708 may also include a power indicator 810 in the form of a logo or other symbol, indicated by the “J” in FIG. 8A. In some embodiments, the power indicator 810 comprises at least one LED and is configured to illuminate to indicate that the light therapy device 600 is powered on. For example, the power indicator 810 may glow red (or any other color) to indicate that the light therapy device 600 is powered on.

FIG. 8B shows the back of the controller 708, including a connector 804. In some embodiments, the connector 804 is a power connector and is configured to receive a cable, plug, or similar mechanism to electrically couple the controller 708 to the flexible PCB assembly 702. Coupling the controller 708 to the flexible PCB assembly 702 also couples the battery 800 to the flexible PCB assembly 702. It should be noted that the light therapy device 600, including the controller 708, may be configured to receive power from an AC or DC power source, rather than the battery 800. The AC or DC power source may also be used to charge the battery 800. Accordingly, the connector 804 may be configured to receive a connection to charge the battery 800, rather than coupling to the flexible PCB assembly 702. In some embodiments, the controller 708 includes at least one recessed area 808, indicated by the dashed boxes in FIG. 8B. The at least one recessed area 808 may be configured to receive a label(s) with product information such as branding, use instructions, product warnings, and the like. The controller 708 may also include a plurality of screws 806 to hold the housing of the controller 708 together. In some embodiments, the housing of the controller 708 is made of ABS plastic and has an ingress protection (IP) 22 rating.

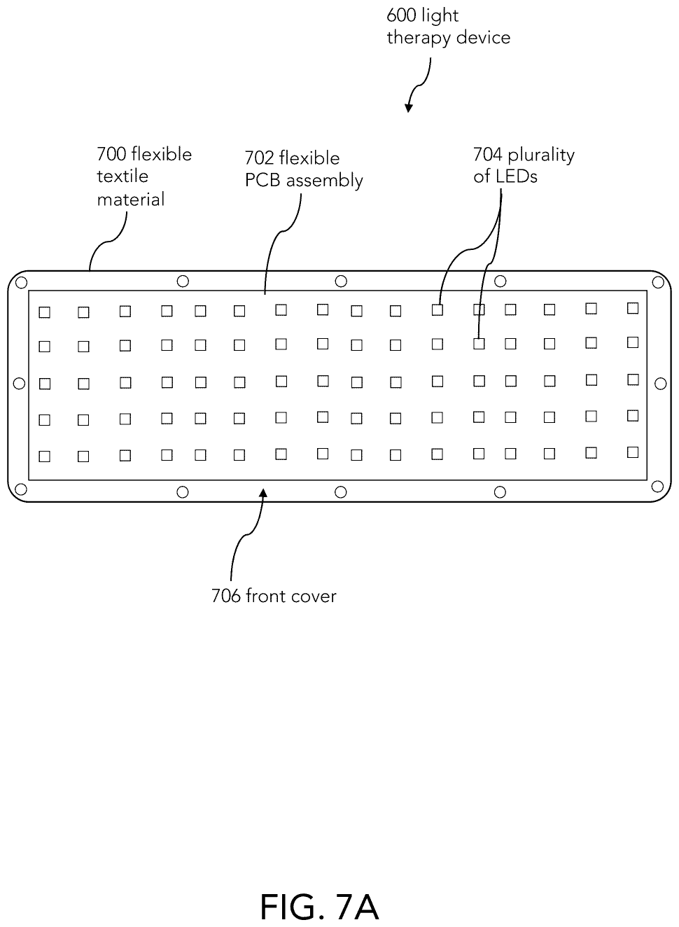

FIGS. 9A and 9B show top and bottom views, respectively, of the controller 708. In some embodiments, the controller 708 includes a plurality of input buttons 900, as shown in FIG. 9A. The plurality of input buttons 900 may include any of the following: a power button 902, a pairing button 904, a start/stop button 906, a mode button 908, and a time button 910. In some embodiments, the power button 902 is configured to move the light therapy device 600 into or out of a standby state.

The pairing button 904 may be configured to enable a user to pair (or unpair) the light therapy device 600 with a third-party device via a local area network (LAN), such as Bluetooth®. In some embodiments, the light therapy device 600 is compatible with a mobile application configured to run on a third-party device such as a smartphone or tablet. The mobile application may operate similarly to the controller 708 in terms of starting or stopping a treatment session and/or selecting treatment time and/or light emission mode. The pairing button 904 may also turn on and/or off pairing for the light therapy device 600. For example, a single press of the pairing button 904 may turn on/off pairing by turning on or off access to the LAN. A press and hold of the pairing button 904 may initiate the pairing process, allowing the light therapy device 600 to “search” for a nearby third-party device for connection. In some embodiments, the controller 708 includes a pairing indicator LED 912 configured to show, via solid light, flashing light, or no light, whether the light therapy device 600 is paired, in the process of pairing, or not paired, respectively, to a third-party device. The pairing indicator LED 912 may be configured to emit blue light.

In some embodiments, the start/stop button 906 is configured to start or stop a light therapy session once the light therapy device 600 is powered on, via the power button 902, out of the standby state. Accordingly, the start/stop button 906 may be configured to start and stop emission of the at least one of red light and NIR light from the plurality of LEDs 704.

The mode button 908 may be configured to select a light emission mode of the plurality of LEDs 704. In some embodiments, the light emission mode selected by the mode button 908 comprises one of red light emission only, NIR light emission only, red and NIR light emission, and a recovery mode, which may also be called a “Recovery Plus Mode.” The light emission modes, including

Recovery Plus Mode, may be the same light emission modes as described with reference to the light therapy device 100. In some embodiments, the controller 708 includes mode indicator LEDs 914, which comprise amber-colored LEDs configured to indicate which light emission mode has been selected. For example, each LED of the mode indicator LEDs 914 may correspond to a different light emission mode, and a user of the light therapy device 600 may be able to cycle through the four options and select a light emission mode by pressing the mode button 908 multiple times.

In some embodiments, the time button 910 is configured to select a treatment time. As shown in FIG. 9A, the controller 708 may include time indicator LEDs 916, where each LED of the time indicator LEDs 916 corresponds to a different predetermined treatment time. The time indicator LEDs 916 may comprise amber-colored LEDs configured to illuminate to indicate the treatment time selected. For example, the time indicator LEDs 916 may correspond to 10, 20, or 30 minute treatment sessions. A user of the light therapy device 600 may be able to cycle through the treatment time options by pressing the time button 910 multiple times.

FIG. 9B shows the bottom of the controller 708, according to some embodiments. As illustrated, the controller 708 may include a first input port 918 and a second input port 920. In some embodiments, the first input port 918 comprises a power input, such as a 24V DC power input. Accordingly, the first input port 918 may be configured to receive a cable to couple the controller 708, including the battery 800, to a DC power source. The first input port 918 may be configured to provide power to the controller 708 to power the flexible PCB assembly 702 and/or to charge the battery 800. The second input port 920 may comprise a Mini-USB port for downloading and/or uploading firmware to the controller 708.

In some embodiments, both the light therapy device 100 and the light therapy device 600 include an auto-shutoff feature. The auto-shutoff may activate following a predetermined time of use, such as 30 minutes. In some embodiments, the auto-shutoff feature is a safety feature designed to protect users in the event that a user falls asleep or otherwise loses track of time while using either the light therapy device 100 or the light therapy device 600. In addition, both the light therapy device 100 and the light therapy device 600 may be configured to be rolled or folded in a specific manner to prevent damage to the flexible PCB assembly 202 and/or the flexible PCB assembly 702.

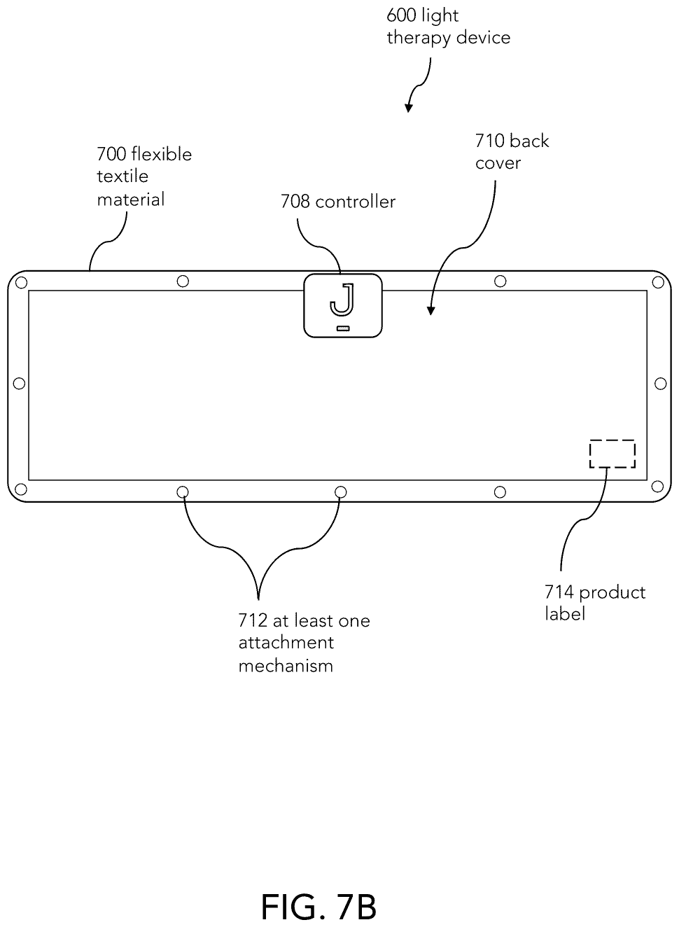

FIG. 10 is a block diagram illustrating aspects of the wearable system disclosed herein. Wearable X2 includes one or more array(s) X4 of light-emitting diodes (LEDs) that produce light in one or more therapeutic wavelengths, such as red light therapy wavelengths. Sensor system X6 is representative of one or more sensors, placed in various locations, on wearable X2 that may be used to monitor a variety of parameters such as wearable temperature, wearable power output, etc. In other embodiments, sensor system X6 detects parameters such as blood pressure, heart rate, etc. Controller X8 may control the functionality of arrays X4 as well as sensor system X6. In some embodiments, this control may be carried out in connection with wireless connectivity, such as by using radio frequency (RF) communication. Controller X8 may be coupled to transceiver X10 via a body area network (BAN). Controller X8 may contain one or more processors or microcontrollers X9. This coupling may be accomplished via a wireless or wired connection between controller X8 and transceiver X10 (hence, the dashed line between the two). In some embodiments, controller X8 may be in communication with transceiver X10 through a paired connection using Bluetooth®, Bluetooth Low Energy (Bluetooth LE), or other local area or wireless personal area network. Transceiver X10 may be, for instance, a mobile device such as a smartphone or tablet. Further, transceiver X10 may transmit and receive information through a terrestrial network or satellite-based network, represented as cloud X12. In some embodiments, transceiver X10 may be implemented as a combined transmitter and receiver unit or as a transmitter and a receiver separate from one another. Data and control information may be received through the cloud X12 by station X14. Station X14, which may have one or more processors (not shown), may serve to monitor information received remotely from the location of wearable X2. In some embodiments, station X14 may both monitor and control functions on wearable X2. This may include controlling the light wavelengths delivered by the arrays X4 of LEDs, power delivered to the LEDs, intensity of individual LEDs, etc. Information received by monitoring station X14 may be saved in storage X16. Consequently, a schedule may be developed and provided in connection with therapeutic treatment using arrays X4.

In some embodiments, LEDs within arrays X4 may provide light at wavelengths between approximately 610 and approximately 850 nanometers (nm). These may include red light at around 660 nm, near-infrared (NIR) light at around 850 nm and light delivered continuously or via pulses using combinations of NIR and red light. In other embodiments, LEDs within arrays X4 may provide a mix of wavelengths, some of which may be considered in the red light spectrum while others are in may be in other spectrum. In other embodiments, some of the LEDs within arrays X4 may be infrared light-emitting diodes (IR LEDs) that emit light in the infrared wavelength range of approximately 700 nm to 1 millimeter (mm).

The wearable system disclosed herein may include a wearable in the form of a garment. For instance, wearable X2 may take to the form of a sweater, vest, cap or band.

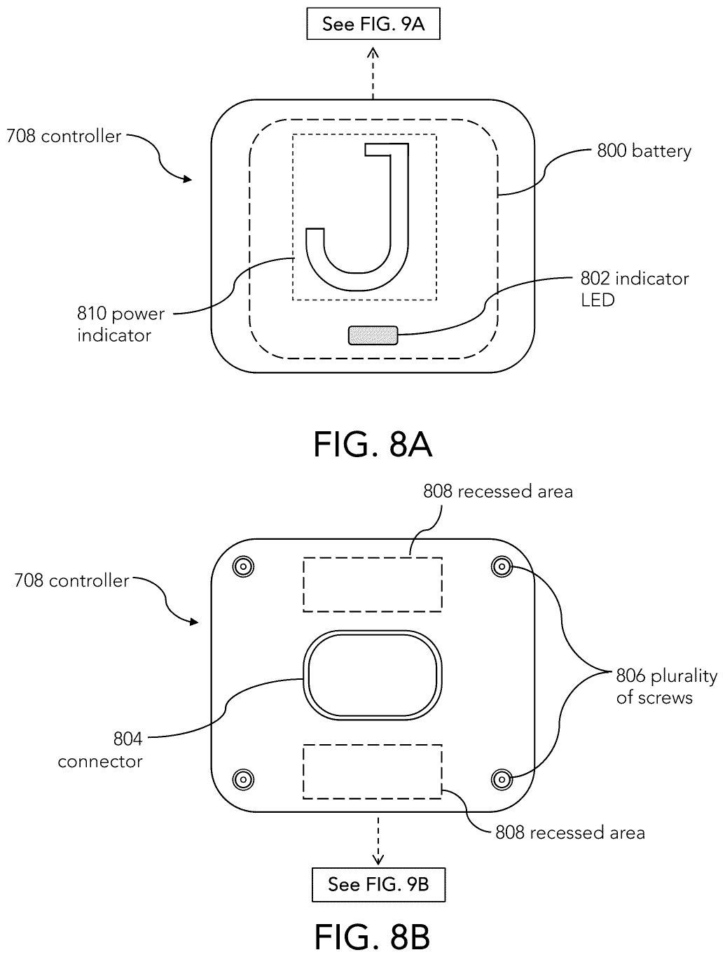

The system discloses herein and shown in FIG. 10 may be further described in connection with FIG. 11. FIG. 11 illustrates a flow chart noting the steps that may be implemented with software, firmware, or other programming in connection with a processor, microcomputer, microcontroller, and the like. With reference to FIG. 11, after the Start step, operating instructions may be received, by a controller, received through merely a keypad, received remotely, or received via a programmed in a device. The wearable including LED functionality and associated sensors may be controlled according to the received instructions. Data may be forwarded, for instance, wirelessly, to a remote location. The data may be used to control functionality on wearable from the remote location. The wearable and associated functionality may be turned off in connection with receiving therapy termination instructions from the remote location or locally at an on-site controller.

The processor(s) noted herein may serve to provide inputs for and or may be used to train artificial intelligence (AI) bots in connection with developing therapies to be carried out on the wearable and its associated functions.

The processor(s) X9 within controller X8 may be used to implement transfer learning in an AI model. Consequently, a specific therapy may be devised for a particular user of wearable X2.

### EXAMPLE

The operating instructions, received and referenced with respect to FIG. 11, by processor(s) X9, may contain information developed pursuant to an AI model that may be developed remotely. The remotely developed AI model may take into a number of inputs that may be received from sensor system X6 or past user operating data that may be recorded and uploaded to cloud X12, such as user body temperature, past therapy application times, desired therapy application times, heart rate, oximeter readings (in embodiments where sensor X6 includes/implements a pulse oximeter), etc.

Transfer learning may be implemented to train an AI model pre-trained through Cloud X12 of FIG. 10 and carried out by processor(s) X9 in order to deliver therapies through wearable X2.

Sensor inputs from sensor X6 may be used to further train the AI model carried out by processor(s) X9.

Some of the components listed herein use the same number from figure to figure. It should be appreciated these components use the same numbers solely for ease of reference and to facilitate comprehension for the reader. While these components may use the same numbers, differences may be present in these components as illustrated in the various figures in which they appear and as described in the specification herein.

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

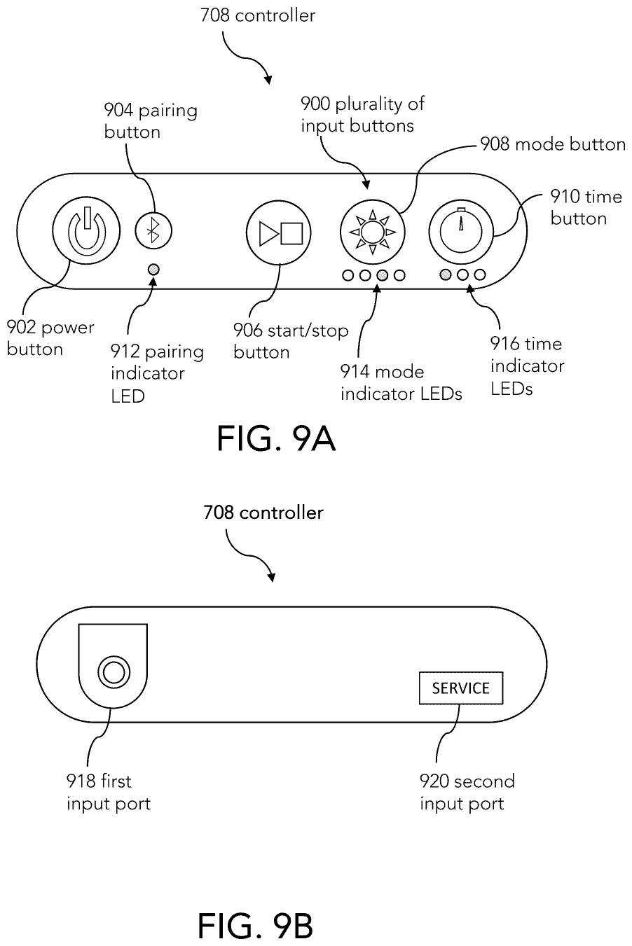

FIG. 12 provides a plan view of a flexible substrate assembly 1200 that fits within the flexible textile material 700 and provides the circuitry to illuminate the plurality of LEDs 704 (not shown in this figure). The flexible substrate assembly 1200 includes a first plurality of red-light traces 1206a-1206n, where “a” equals one and “n” equals any number greater than one. The first plurality of red-light traces 1206a-1206n is energized by a red-light connector pad 1204a that is coupled to a conductor 1202a. The red-light connector pad 1204a, in accordance with one embodiment, is a have of a snap-type connector, where the two halves provide a compression coupling that retain one another once connected. On the opposing end of the first plurality of red-light traces 1206a-1206n is a corresponding conductor 1210a coupled to a connector pad 1211a. The connector pad 1211a can also be a half of another snap pair. Each of the red-light contact pad 1204a, conductor 1202a, the first plurality of red-light traces 1206a-1206n, the conductor 1210a, and red-light contact pad 1211a are made of electrically conductive material. Each of the contact pad 1204a, conductor 1202a, the first plurality of red-light traces 1206a-1206n, the conductor 1210a, and contact pad 1211a have small electrically insulating gaps between them. The contact pad 1204a, conductor 1202a, the first plurality of red-light traces 1206a-1206n, the conductor 1210a, and contact pad 1211a comprise a first trace group 1201a.

A first plurality of infrared-light traces 1208a-1208n runs parallel to and in close proximity to the first plurality of red-light traces 1206a-1206n. The first plurality of infrared-light traces 1208a-1208n is energized by an infrared-light connector pad 1203a that is coupled to a conductor 1202b. On the other end of the first plurality of infrared-light traces 1208a-1208n is a corresponding conductor 1213a coupled to an infrared-light connector pad 1212a. Each of the contact pad 1203a, conductor 1205a, the first plurality of infrared-light traces 1208a-1208n, the conductor 1210b, and contact pad 1211b are also made of electrically conductive material. Each of the contact pads 1203a, conductor 1202a, the first plurality of red-light traces 1206a-1206n, the conductor 1210a, and contact pad 1211a have small electrically insulating gaps between them. The contact pad 1203a, the conductor 1205a, the first plurality of infrared-light traces 1208a-1208n, the conductor 1210b, and contact pad 1211b comprise a second trace 1201b group.

The flexible substrate assembly 1200 shown in FIG. 12 includes a plurality of additional groups of red-light traces and infrared-light traces 1201c-n to create an entire array of conductive rows that can be used to energize an array of LED lights. In FIG. 12, the first red-light trace group 1201a-n can be energized by placing an electric potential between contacts 1204a-1204n on the one side and corresponding contacts 1211a-1211n on the other. Similarly, each infrared-light trace group 1201b-n can be energized by placing an electric potential between contacts 1203a-1203n on one side and corresponding contacts 1212a-1212n on the other. When the LEDs 704 are placed across the gaps between the traces 1206a-n and in conductive contact with the traces 1206a-n on either side of the LEDs 704, current is able to pass from one trace 1206 to another and illuminate the LEDs 704. This configuration creates a standard LED circuit, as is well known to those of ordinary skill in the art, but the instant circuit is in a novel flexible format. In one embodiment, the flexible substrate assembly 1200 is made of a nonwoven coated substrate material. The coating protects and insulates the trace groups 1201 and corresponding contact pads.

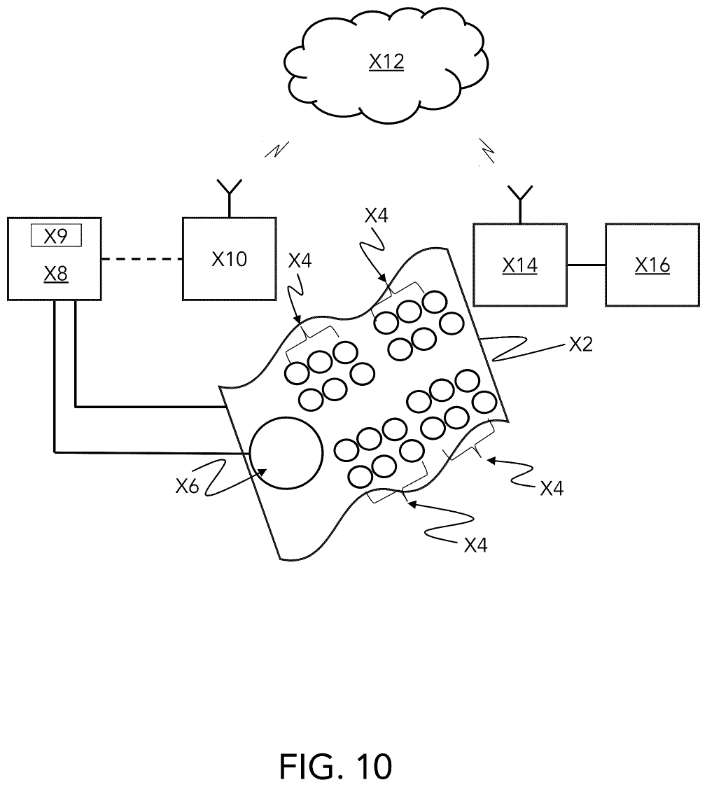

FIG. 13 shows a flexible textile cable assembly 1300 that provides power to the flexible substrate assembly 1200. The flexible textile cable assembly 1300 includes a first anchor strip 1301 and a second anchor strip 1303. The first anchor strip 1301 features a first plurality of conductive red-light snaps 1302a-n that each are configured to mate with a corresponding one of the red-light connector pads 1204a-n shown in FIG. 12. The second anchor strip 1303 features a second plurality of conductive red-light snaps 1318a-n that each are configured to mate with a corresponding one of the opposing red-light connector pads 1211a-n shown in FIG. 12. Similarly, the first anchor strip 1301 features a first plurality of conductive infrared-light snaps 1304a-n that are each configured to mate with a corresponding one of the infrared-light connector pads 1203a-n shown in FIG. 12. The second anchor strip 1303 features a second plurality of conductive infrared-light snaps 1316a-n that each are configured to mate with a corresponding one of the opposing infrared-light connector pads 1212a-n shown in FIG. 12.

The term “snap” or “snaps,” as used herein, is intended to indicate any type of connector that electrically couples two conductive materials to one another. One specific embodiment is a metallic snap, such as those used on clothing, but the invention is in no way limited to any specific embodiment and can include other connectors as well.

Each of the first plurality of conductive red-light snaps 1302a-n is coupled to and energized by a corresponding one of a plurality of non-illustrated conductive wires that, for example, provides a positive potential to the first plurality of conductive red-light snaps 1302a-n. Each of the second plurality of conductive red-light snaps 1318a-n is coupled to a corresponding one of a plurality of non-illustrated conductive wires that, for example, provides a negative potential to the second plurality of conductive red-light snaps 1318a-n.

Similarly, each of the first plurality of conductive infrared-light snaps 1304a-n is coupled to and energized by a corresponding one of a plurality of non-illustrated conductive wires that, for example, provide a positive potential to the first plurality of conductive infrared-light snaps 1304a-n. Each of the second plurality of conductive infrared-light snaps 1316a-n is coupled to a corresponding one of a plurality of non-illustrated conductive wires that, for example, provides a negative potential to the second plurality of conductive infrared-light snaps 1316a-n.

The first anchor strip 1301 features a first plurality of non-conductive snaps 1306a-n and the second anchor strip 1303 features a second plurality of conductive non-conductive snaps 1320a-n. The first plurality of non-conductive snaps 1306a-n and the second plurality of conductive non-conductive snaps 1320a-n are used to secure an electrically insulating cover of the flexible textile cable assembly 1300.

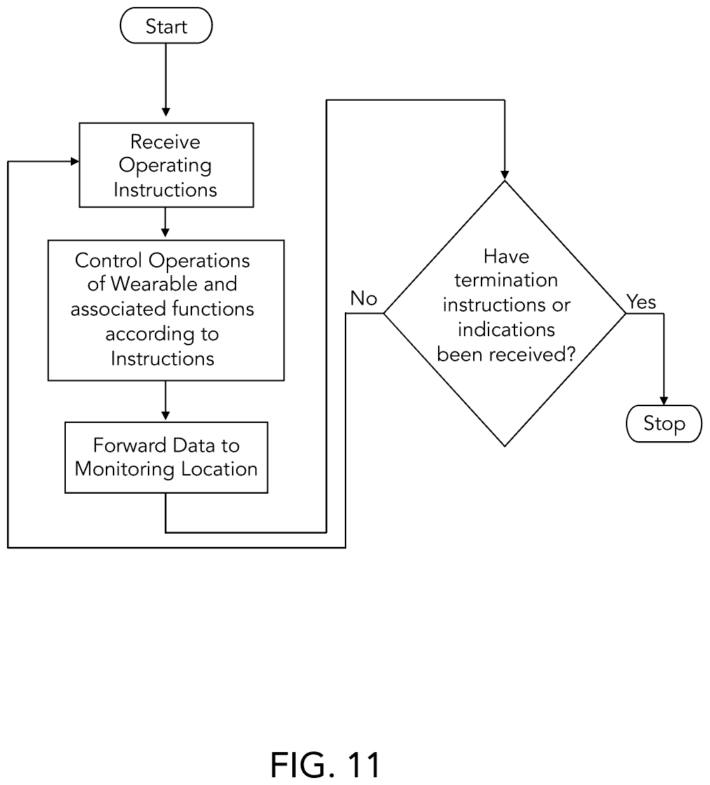

A connector strip 1305 physically connects the first anchor strip 1301 to the second anchor strip 1303. The connector strip 1305 couples to the first anchor strip 1301 via a first group of snaps 1308a-n and couples to the second anchor strip 1303 via a second group of snaps 1314a-n. The connector strip 1305 has along its length a plurality of non-conductive snaps 1312a-n. Along with non-conductive snaps 1306a-n and 1320a-n, non-conductive snaps 1312a-n provide secure anchors to couple a cover 1402 (shown in FIG. 14) to the flexible textile cable assembly 1300. Not only does the cover 1402 physically protect the flexible textile cable assembly 1300, it also provides an electrically insulating barrier between a user of the device and the electrically charged connector pads 1302a-n, 1304a-n, 1316a-n, and 1318a-n.

FIG. 14 provides a perspective partial view of the flexible textile cable assembly 1300 partially coupled to the flexible substrate assembly 1200. In this view, connector strip 1305 is attached to a flexible bottom cover 1401, e.g., by snap connectors or by sewing the connector strip 1305 to the flexible bottom cover 1401. The flexible substrate assembly 1200 is positioned upon the flexible bottom cover so that the trace groups 1201a-n run longitudinally parallel to the longitudinal direction of the connector strip 1305.

The second anchor strip 1303 is shown having a portion of its red-light snaps 1318a-n coupled to a corresponding portion of the red-light connector pads 1211a-n of the flexible substrate assembly 1200. Likewise, the second anchor strip 1303 is shown having a portion of its infrared-light snaps 1316a-n coupled to a corresponding portion of the infrared-light connector pads 1212a-n of the flexible substrate assembly 1200.

FIG. 14 shows that a portion of the second anchor strip 1303 is not yet attached to the remaining portion of the flexible substrate assembly 1200. This part of the figure shows the snap halves that form the physical connection between the electrically conducting contacts. Specifically, five red-light connector pads 1211a-e are shown and are spaced to mate with five (only four can be seen in this view) corresponding snap couples 1318a-e. Similarly, five infra-red light connector pads 1212a-e are shown and are spaced to mate with five (only four can be seen in this view) corresponding snap couples 1316a-e.

Each snap 1316a-n and 1318a-n is a terminal end of a non-illustrated wire that runs through the anchor strip 1303. Similarly, each snap 1302a-n and 1304a-n is a terminal end of a non-illustrated wire that runs through anchor strip 1301. When an electrical potential is applied across the corresponding snap pairs, the electric potential energizes each trace group 1201. For example, if a wire having a positive charge is coupled to snap 1302a, that charge is transferred to contact pad 1204a. If a wire having a negative charge, or ground, is coupled to snap 1318a, that charge, or ground connection, is applied to snap 1318a. Through these wires and charges, electric current can be applied to trace group 1201a. If an LED is coupled between each one of the plurality of light traces 1206a-n, the current will energize each of the LEDs. FIG. 14 shows a plurality of LEDs 1404a-n coupled in such a manner between the plurality of traces 1206a-n.

A second and different electrical potential can be applied between connectors pads 1203a and 1212a through snaps 1304a and 1316a. Through these wires and charges, electric current can be applied to trace group 1201b. If an LED is coupled between each one of the plurality of light traces 1206a-n, the current will energize each of the LEDs. FIG. 14 shows a plurality of LEDs 1404a-n coupled in such a manner between the plurality of traces 1206a-n. By placing a different electric potential across trace group 1201a than 1201b, the LEDs 1404a-n can be made to radiate different colors, specifically, red light and infrared light. When the entire flexible substrate assembly 1200 is populated with LEDs 1404, a panel of LEDs is provided that produces a user-selectable output of light.

The connector strip 1305 electrically couples to the second anchor strip 1303 when the snap halves 1314a-d on the connector strip 1305 are fixedly mechanically coupled to the corresponding snap halves 1402a-d on the anchor strip 1303. This connection allows the current to return, via connector strip 1305, to the power source (discussed below).

The anchor strips 1301 and 1303 and the connector strip 1305 are, in one embodiment, made of a cloth material that provides flexibility to the overall assembly as well as electrical insulation for/from all elements carrying a charge. Once all the above-described snap connectors are coupled to one another, the only thing exposed to view and to the touch of the user of the device are the LEDs 1404.

FIG. 13 shows a power source 1310 that powers the connectors 1302a-n and 1304a-n through one of its terminals and connectors 1316a-n and 1318a-n through the other of its two terminals. The power source 1310 can be any conventional source of electric power, including battery or wall-supplied power. Preferably, the power source 1310 is a direct current (DC) power source that provides direct current to the plurality of LEDs 1404.

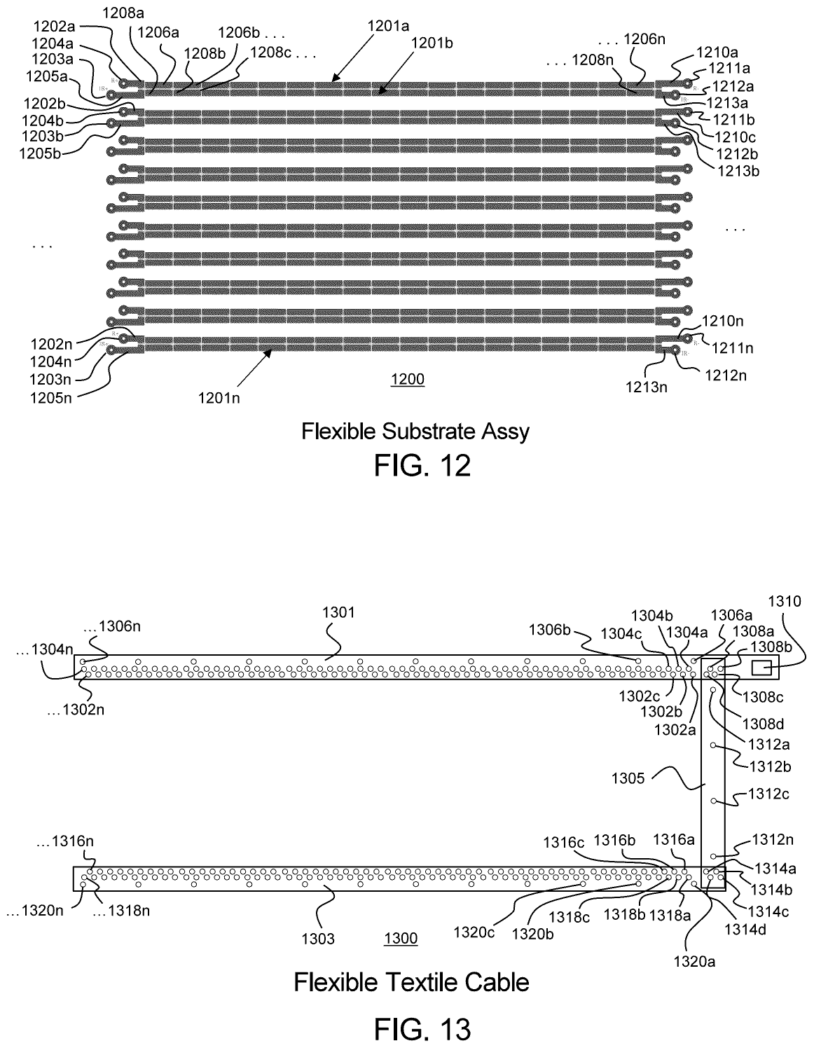

FIG. 15 shows a fully assembled flexible light-emitting blanket assembly 1500. It includes a flexible textile material cover 1504 that is made of a back cover portion 1502 and a front cover portion 1506. The use of the terms “front” and “back” is intended to differentiate two portions of the flexible textile material 1504 and is not intended to indicate that one necessarily has to be above, below, in front of, or behind the other, with reference to any other object. In one embodiment, the front portion 1506 of the flexible textile material 1504 is an opaque material that allows light emitted from the plurality of LEDs 1404a-n to pass through the front cover portion 1506 of the flexible textile material cover 1504. The front cover portion 1506 of the flexible textile material cover 1504 can be made of a textile material that wicks away sweat from the user and provides some isolation of heat between the plurality of LEDs 1404a-n and the user upon whose skin the flexible light-emitting blanket assembly 1500 is placed. In accordance with one embodiment, the back cover portion 1502 of the flexible textile material cover 1504 is made of a material with metallic materials, e.g., aluminum, allowing it to dissipate heat in an efficient manner, acting as a type of heat sink.

In one embodiment, the flexible textile material 1504 of the flexible light-emitting blanket assembly 1500 has a length and width large enough to cover the torso of an average human. For example, it is estimated that the average height of a man is 5.6 ft and the average height of a woman is 5.2 ft. Thus, the flexible textile material 1504 would be approximately 3.5 ft in length and 2.5 ft in width. Within the flexible light-emitting blanket assembly 1500, there are no structures that will prevent the flexible light-emitting blanket assembly 1500 from flexing across the length and width of the flexible textile material to conform to the shape of the torso of the average human.

Advantageously, the flexible textile material cover 1504 can be removed for cleaning. Regular cleaning may be desired because, in use, portions of the flexible light-emitting blanket assembly 1500 rest against the user's skin and sweat will transfer to those portions. For this reason, the cover 1504 is conveniently removable via the non-conductive snaps 1306a-n, 1312a-n, and 1320a-n, all shown in FIG. 15. The entire cover 1504 or portions of the cover 1504, e.g., upper portion 1506, can be made of any flexible textile material that is strong enough to secure the flexible substrate assembly 1200 and the flexible textile cable assembly 1300 and also easily launders clean.

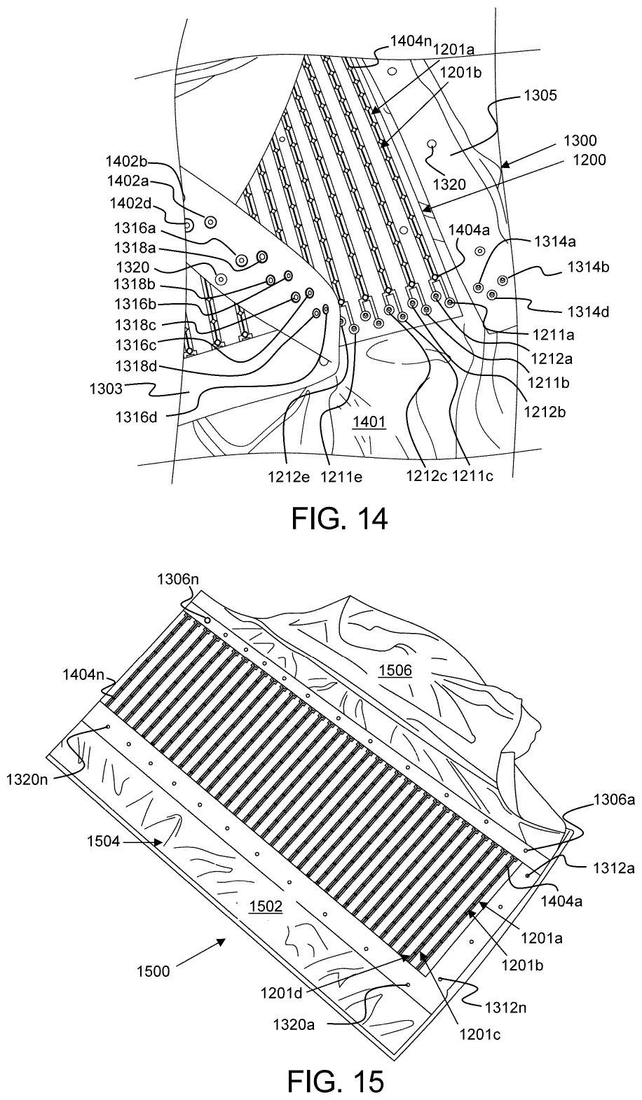

FIG. 16 shows the internal wiring of an exemplary one of the anchor strips 1301 or 1303. In one embodiment, each anchor strip is made of a pair of fabric layers that obscure wire pairs 1602, 1604 and 1606, 1608. In FIG. 16, one of the fabric layers has been removed to expose the wire pairs 1602, 1604 and 1606, 1608. A first wire pair 1602 and 1604 are electrically coupled to each of the red-light snaps 1318a-n. Similarly, the second wire pair 1606 and 1608 are electrically coupled to each of the infrared-light snaps 1316a-n. The wire pairs 1602, 1604 and 1606, 1608 allow an electrical potential to be selectively applied to the red-light snaps 1318a-n and the infrared-light snaps 1316a-n.

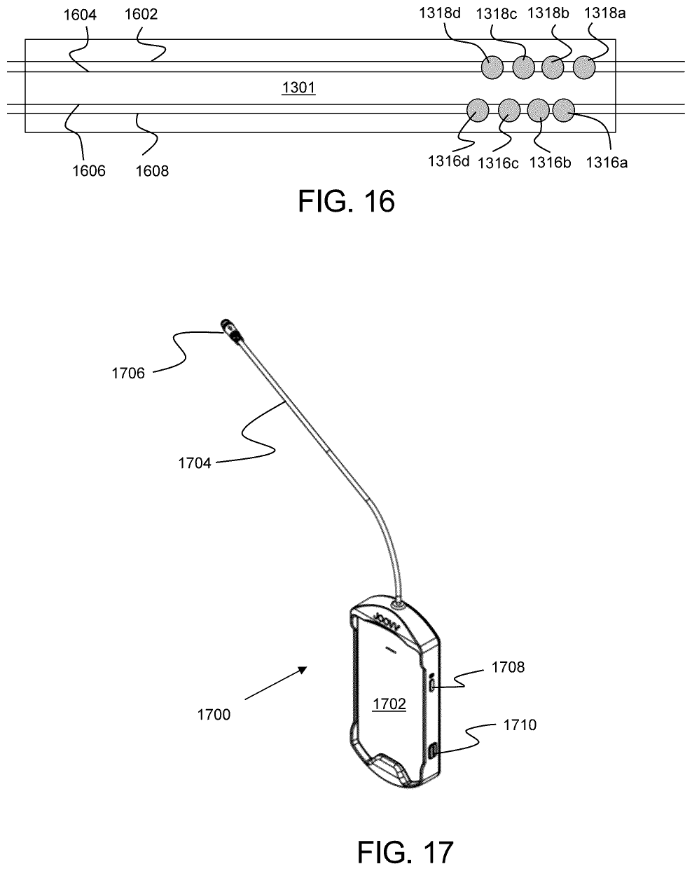

FIG. 17 shows a docking station 1700 that is configured to physically receive and communicatively interface with the controller 300. The docking station 1700 has a cradle portion 1702 that mates with and holds the controller 300. The docking station 1700 has a cable 1704 with a docking station connector 1706 that communicatively couples with the nonwoven coated substrate assembly 1200. When the connector 1706 is coupled to the substrate assembly 1200 and the controller 300 is in the cradle portion 1702, the controller 300 controls the flexible light-emitting blanket assembly 1500 in the way described above (where the flexible light-emitting blanket assembly 1500 is more generally referred to as “the light therapy device 100”). Advantageously, the docking station 1700 allows the controller 300 to be removed for charging in a separate base. In this way, a battery (or batteries) within the controller 300 can be charged at a separate charging device and then returned to the cradle portion 1702 to power and control the flexible light-emitting blanket assembly 1500. The docking station 1700 also includes a power button 1708 configured to enter and exit a standby state and a pairing button 1710 configured to turn on and off a local area network pairing function that allows it to connect to a third-party device.

Advantageously, the present invention introduces the first fully flexible light-emitting blanket assembly that is entirely free of any interior structure which might inhibit its ability to conform to the unique contours of a user's body. As a result, the light-emitting blanket not only offers superior adaptability and comfort by conforming to the body's curves, but it also maintains its functional integrity without sacrificing flexibility. This breakthrough addresses a significant shortcoming in the existing art, marking a pivotal advancement in the design and utility of light-emitting blanket assemblies.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section. The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

The foregoing may be accomplished through software code running in one or more processors on a communication device in conjunction with a processor in a server running complementary software code.

Some of the devices, systems, embodiments, and processes use computers. Each of the routines, processes, methods, and algorithms described in the preceding sections may be embodied in, and fully or partially automated by, code modules executed by one or more computers, computer processors, or machines configured to execute computer instructions. The code modules may be stored on any type of non-transitory computer-readable storage medium or tangible computer storage device, such as hard drives, solid state memory, flash memory, optical disc, and/or the like. The processes and algorithms may be implemented partially or wholly in application-specific circuitry. The results of the disclosed processes and process steps may be stored, persistently or otherwise, in any type of non-transitory computer storage such as, e.g., volatile or non-volatile storage.

It is appreciated that in order to practice the method of the foregoing as described above, it is not necessary that the processors and/or the memories of the processing machine be physically located in the same geographical place. That is, each of the processors and the memory (or memories) used by the processing machine may be located in geographically distinct locations and connected so as to communicate in any suitable manner. Additionally, it is appreciated that each of the processor and/or the memory may be composed of different physical pieces of equipment. Accordingly, it is not necessary that the processor be one single piece of equipment in one location and that the memory be another single piece of equipment in another location. That is, it is contemplated that the processor may be two pieces of equipment in two different physical locations. The two distinct pieces of equipment may be connected in any suitable manner. Additionally, the memory may include two or more portions of memory in two or more physical locations.

To explain further, processing, as described above, is performed by various components and various memories. However, it is appreciated that the processing performed by two distinct components as described above may, in accordance with a further embodiment of the foregoing, be performed by a single component. Further, the processing performed by one distinct component as described above may be performed by two distinct components. In a similar manner, the memory storage performed by two distinct memory portions, as described above, may, in accordance with a further embodiment of the foregoing, be performed by a single memory portion. Further, the memory storage, performed by one distinct memory portion, as described above, may be performed by two memory portions.

Further, various technologies may be used to provide communication between the various processors and/or memories, as well as to allow the processors and/or the memories of the foregoing to communicate with any other entity, i.e., so as to obtain further instructions or to access and use remote memory stores, for example. Such technologies used to provide such communication might include a network, the Internet, Intranet, Extranet, LAN, an Ethernet, wireless communication via cell tower or satellite, or any client server system that provides communication, for example. Such communications technologies may use any suitable protocol such as TCP/IP, UDP, or OSI, for example.

As described above, a set of instructions may be used in the processing of the foregoing. The set of instructions may be in the form of a program or software. The software may be in the form of system software or application software, for example. The software might also be in the form of a collection of separate programs, a program module within a larger program, or a portion of a program module, for example. The software used might also include modular programming in the form of object-oriented programming. The software may instruct the processing machine what to do with the data being processed.

Further, it is appreciated that the instructions or set of instructions used in the implementation and operation of the foregoing may be in a suitable form such that the processing machine may read the instructions. For example, the instructions that form a program may be in the form of a suitable programming language, which is converted to machine language or object code to allow the processor or processors to read the instructions. That is, written lines of programming code or source code, in a particular programming language, are converted to machine language using a compiler, assembler or interpreter. The machine language is binary coded machine instructions that are specific to a particular type of processing machine, i.e., to a particular type of computer, for example. The computer understands the machine language.

Any suitable programming language may be used in accordance with the various embodiments of the foregoing. Illustratively, the programming language used may include assembly language, Ada, APL, Basic, C, C++, COBOL, dBase, Forth, Fortran, Java, Modula-2, Pascal, Prolog, Python, REXX, Visual Basic, and/or JavaScript, for example. Further, it is not necessary that a single type of instruction or single programming language be utilized in conjunction with the operation of the system and method of the foregoing. Rather, any number of different programming languages may be utilized as is necessary and/or desirable.

Also, the instructions and/or data used in the practice of the foregoing may utilize any compression or encryption technique or algorithm, as may be desired. An encryption module might be used to encrypt data. Further, files or other data may be decrypted using a suitable decryption module, for example.

As described above, the foregoing may illustratively be embodied in the form of a processing machine, including a computer or computer system, for example, that includes at least one memory. It is to be appreciated that the set of instructions, i.e., the software for example, that enables the computer operating system to perform the operations described above may be contained on any of a wide variety of media or medium, as desired. Further, the data that is processed by the set of instructions might also be contained on any of a wide variety of media or medium. That is, the particular medium, i.e., the memory in the processing machine, utilized to hold the set of instructions and/or the data used in the foregoing may take on any of a variety of physical forms or transmissions, for example. Illustratively, the medium may be in the form of paper, paper transparencies, a compact disk, a DVD, an integrated circuit, a hard disk, a floppy disk, an optical disk, a magnetic tape, a RAM, a ROM, a PROM, an EPROM, a wire, a cable, a fiber, a communications channel, a satellite transmission, a memory card, a SIM card, or other remote transmission, as well as any other medium or source of data that may be read by the processors of the foregoing.

Further, the memory or memories used in the processing machine that implements the foregoing may be in any of a wide variety of forms to allow the memory to hold instructions, data, or other information, as is desired. Thus, the memory might be in the form of a database to hold data. The database might use any desired arrangement of files such as a flat file arrangement or a relational database arrangement, for example.

In the system and method of the foregoing, a variety of “user interfaces” may be utilized to allow a user to interface with the processing machine or machines that are used to implement the foregoing. As used herein, a user interface includes any hardware, software, or combination of hardware and software used by the processing machine that allows a user to interact with the processing machine. A user interface may be in the form of a dialogue screen for example. A user interface may also include any of a mouse, touch screen, keyboard, keypad, voice reader, voice recognizer, dialogue screen, menu box, list, checkbox, toggle switch, a pushbutton or any other device that allows a user to receive information regarding the operation of the processing machine as it processes a set of instructions and/or provides the processing machine with information. Accordingly, the user interface is any device that provides communication between a user and a processing machine. The information provided by the user to the processing machine through the user interface may be in the form of a command, a selection of data, or some other input, for example.

As discussed above, a user interface is utilized by the processing machine that performs a set of instructions such that the processing machine processes data for a user. The user interface is typically used by the processing machine for interacting with a user either to convey information or receive information from the user. However, it should be appreciated that in accordance with some embodiments of the system and method of the foregoing, it is not necessary that a human user actually interact with a user interface used by the processing machine of the foregoing. Rather, it is also contemplated that the user interface of the foregoing might interact, i.e., convey and receive information, with another processing machine, rather than a human user. Accordingly, the other processing machine might be characterized as a user. Further, it is contemplated that a user interface utilized in the system and method of the foregoing may interact partially with another processing

machine or processing machines, while also interacting partially with a human user. While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein

The claims appended hereto are meant to cover all modifications and changes within the scope and spirit of the present invention.

What is claimed is:

1. A light therapy device, comprising:
a flexible textile material having a length and width large enough to cover a torso of an average human;
a flexible substrate assembly coupled to and interior of the flexible textile material;
a flexible textile cable assembly coupled to an interior of the flexible textile material; and
a plurality of light emitting diodes (LEDs) electrically coupled to the flexible substrate assembly, wherein the plurality of LEDs is arranged and configured to emit at least one of red light and near-infrared light,
wherein the light therapy device is void of structures that prevent the light therapy device from flexing across the length and width of the flexible textile material to conform to the shape of the torso of the average human.

2. The light therapy device of claim 1, wherein the flexible textile material comprises:
at least one attachment mechanism removably coupling the flexible substrate assembly and the flexible textile cable assembly to the interior of the flexible textile material.

3. The light therapy device of claim 2, wherein the at least one attachment mechanism is a non-conductive snap structure.

4. The light therapy device of claim 1, wherein the flexible textile material comprises:
a front cover of a semi-transparent material configured to diffuse at least a portion of the at least one of red light and near-infrared light emitted by the plurality of LEDs.

5. The light therapy device of claim 1, wherein the flexible textile material is at least partially metallic and is capable of diffusing heat generated by the plurality of LEDs.

6. The light therapy device of claim 1, further comprising:
a controller electrically coupled to the flexible textile cable assembly and the flexible substrate assembly, configured to control emission of the at least one of red light and near-infrared light from the plurality of LEDs, the controller having:
a select mode button configured to select a light emission mode of the plurality of LEDs;
a help button configured to open up help menus;
a settings button configured to open up a settings menu;
a play button configured to start/stop/pause emission mode of the plurality of LEDs; and
a time button configured to select a treatment time.

7. The light therapy device of claim 6, wherein the light emission mode comprises preset modes and a custom mode selection, wherein the preset modes include red and near-infrared light in continuous and pulsed waveform at preset time durations and where the custom mode selection includes red and near-infrared light in continuous and pulsed waveform at customizable time durations.

8. The light therapy device of claim 7, wherein the recovery mode comprises:
substantially continuous red-light emission with pulsed near-infrared light emission.

9. The light therapy device of claim 7, wherein the controller comprises:
a power button configured to at least one of enter and exit a standby state; and
a pairing button configured to at least one of turn on and off local area network pairing with a third-party device.

10. The light therapy device of claim 1, further comprising:
a connector coupled to the flexible textile material and the textile cable assembly, wherein the textile cable assembly is electrically coupled to the substrate assembly and the connector is configured to receive a cable coupled to a controller docking station, thereby electrically coupling the controller docking station to the nonwoven coated substrate assembly.

11. The light therapy device of claim 1, further comprising:
a controller electrically coupled to the flexible textile cable assembly and the flexible substrate assembly, configured to control emission of the at least one of red light and near-infrared light from the plurality of LEDs, the controller having a display screen configured to display information indicating at least one of treatment time, light emission mode, local area network pairing status, and treatment start/stop status.

12. The light therapy device of claim 1, wherein the flexible PCB assembly comprises:
a nonwoven coated substrate;
a plurality of flexible printed circuit boards (PCBs) on the nonwoven coated substrate; and
a plurality of electrically conductive connectors each attached to one of the plurality of flexible PCBs.

13. The light therapy device of claim 1, further comprising:
a controller electrically coupled to the flexible textile cable assembly and the flexible substrate assembly, configured to control emission of the at least one of red light and near- infrared light from the plurality of LEDs and having a built-in rechargeable battery configured to provide power to the light therapy device.

14. A wearable LED treatment system comprising:
a flexible substrate assembly;
a flexible textile cable assembly physically and electrically coupled to the flexible substrate assembly and having a length and width large enough to cover a torso of an average human;
a plurality of light emitting diodes (LEDs) physically and electrically coupled to the flexible substrate assembly, wherein the plurality of LEDs is arranged and configured to emit at least one of red light and near-infrared light;
a power source electrically coupled to the flexible textile cable assembly; and
a flexible textile cover including:
a front panel having a semi-transparent portion configured to diffuse at least a portion of the at least one of red light and near-infrared light emitted by the plurality of LEDs; and
a back panel with fasteners configured to removably couple and decouple the flexible textile cable assembly to the flexible textile cover,
wherein the wearable LED treatment system is void of structures that prevent the light therapy device from flexing across the length and width of the flexible textile material to conform to the shape of the torso of the average human.

15. The wearable LED treatment system of claim 14, wherein the back panel is made of a metallic material and is capable of diffusing heat generated by the plurality of LEDs

16. The wearable LED treatment system of claim 14, wherein the fasteners are non-conductive snap structures.

17. The wearable LED treatment system of claim 14, further comprising:
a controller electrically coupled to the flexible textile cable assembly and the flexible substrate assembly, configured to control emission of the at least one of red light and near-infrared light from the plurality of LEDs

18. The wearable LED treatment system of claim 17, further comprising:
a transceiver configured to couple the controller to a wireless network.

19. The wearable LED treatment system of claim 18, wherein the transceiver is included in a smartphone or a tablet.

20. The wearable LED treatment system of claim 14, wherein ones of the plurality of LEDs are operable to produce light at wavelengths selected from the group consisting of light at a wavelength of between about 630 nanometers and 660 nanometers, light at a wavelength of between about 800 nanometers and 900 nanometers, light at a wavelength of between about 600 nanometers and 750 nanometers, light at a wavelength of between about 900 nanometers and 1 millimeter and a combination thereof.
